(19) United States
(12) Patent Application Publication
(10) Pub. No.: US 2026/0044959 A1
(43) Pub. Date: Feb. 12, 2026

(54) Automated Lesion Detection

(71) Applicant: LightLab Imaging, Inc., Westford, MA (US)

(72) Inventor: Shen Liu, Brooklyn, NY (US)

(73) Assignee: LightLab Imaging, Inc., Westford, MA (US)

(21) Appl. No.: 19/101,341

(22) PCT Filed: May 15, 2024
(86) PCT No.: PCT/US2024/029469

§ 371 Date: Feb. 5, 2025

Related U.S. Application Data
(60) Provisional application No. 63/527,108, filed on Jul. 17, 2023.

(51) Int. Cl. G06T 7/00; A61B 5/02; G06V 10/25

(52) U.S. Cl. CPC G06T 7/0014; A61B 5/02007; G06V 10/25; G06T 2207/30096; G06T 2207/30101; G06V 2201/03

(57) ABSTRACT

The present disclosure provides systems and methods for determining an external elastic lamina (“EEL”) value for intravascular image frames and using the predicted EEL to determine a plaque burden for respective image frames. The predicted EEL value may be determined based on a threshold number of image frames within a region of interest having plaque burden below a threshold plaque burden. In some examples, the predicted EEL value may be determined as a function of intima thickness and media thickness and the H-K model derived expected lumen diameter. The determined plaque burden and visible EEL arc may be used to automatically identify lesions and suggest candidate treatment zones.

### CROSS-REFERENCE TO RELATED APPLICATIONS

This application claims the benefit of the filing date of U.S. Provisional Patent Application No. 63/527,108 filed Jul. 17, 2023, the disclosure of which is hereby incorporated herein by reference.

### BACKGROUND

Various measurements of a blood vessel may be automatically determined using computer software, based on image data captured during a pullback of an intravascular probe within the blood vessel. However, the presence of plaque or other buildup in the blood vessel can prevent or make more difficult the automatic determination of such measurements. This may result in incomplete data about the blood vessel being obtained from the pullback. Incomplete data may result in poor further determinations using such data, for example the automatic selection of candidate treatment zones, or may prevent such further determinations from being made automatically altogether. This may result in a physician having to manually enter or adjust clinical parameters such as candidate treatment zones.

### BRIEF SUMMARY

The disclosure is generally directed to apparatus, methods, computer program code and so forth for automatically estimating external elastic lamina (“EEL”) value(s) for intravascular image frames. The estimated EEL can be used to determine a plaque burden for image frames that the EEL value was estimated for. The EEL value may be, for example, the EEL diameter or the EEL area. For example, plaque may have overgrown a vessel such that the EEL value cannot be automatically determined during a pullback or visible to a user, such as an analyst or a physician. For the image frames where the EEL value is not detected and/or erroneously measured, the EEL value may be predicted. The EEL value may be predicted for image frames within a region of interest. The region of interest may be, for example, a region of the vessel between two side branches.

In one example, the region of interest may include a first set of frames and a second set of frames. The first set of frames may be image frames in which the EEL value was detected during a pullback. The second set of frames may be image frames in which the EEL value was not detected during a pullback. If the first set of frames includes a threshold number of image frames having a plaque burden below a threshold plaque burden, at least the threshold number of frames may be used to predict the EEL value. The threshold plaque burden may be, for example, a healthy plaque burden. The threshold plaque burden may be a plaque burden between about 0-50%. A representative value of the EEL values of the threshold number of image frames may be determined. The representative EEL value may be, for example, an average, median, or mean EEL value. The representative EEL value may be used as the predicted EEL value for the image frames in the second set of frames. According to some examples, the representative EEL value of the first set of frames may be used as the determined EEL value for frames in the first subset of frames. For example, if some of the frames in the first subset of frames have erroneously measured EEL values, the determined representative EEL value may be used as the EEL value for the frames in the first subset of frames having an erroneous EEL value. The erroneous EEL value may be an EEL value that is beyond a standard deviation of the determined representative EEL value, that is outside a predetermined range of the determined representative EEL value, or the like.

In another example, the EEL value for frames within the region of interest may be predicted as a function of intima thickness and media thickness. Continuing with the previous example, if the first set of frames does not include the threshold number of frames having a plaque burden below a threshold plaque burden, the EEL value may be predicted based on the intima thickness and media thickness. In some examples, the EEL value may be further based on a H-K model. According to some examples, the EEL value may be predicted based on the intima thickness, media thickness, and the H-K model. According to some examples, if the EEL value in a segment of the vessel distal to the region of interest is larger than the predicted EEL value for the region of interest, the EEL value of the distal segment may be used as the predicted EEL value for the region of interest. The EEL value of the distal segment may be a representative EEL value of the distal segment. In some examples, if the EEL value in a segment of the vessel proximal to the region of interest is less than the predicted EEL value of the region of interest, the EEL value of the proximal segment may be used as the predicted EEL value for the region of interest. The EEL value of the proximal segment may be a representative EEL value of the proximal segment. The EEL value of the proximal or distal segment may be used based on the natural taper of a vessel from a proximal vessel segment to a distal vessel segment.

The predicted EEL values may be used to determine a plaque burden for respective image frames. For example, the predicted EEL value and the lumen area for a given frame may be used to determine the plaque burden. The determined plaque burden may be used to identify lesions within the vessel. According to some examples, if a first lesion is within a threshold distance from a second lesion, the first and second lesion may be identified as a combined lesion. The identified lesions may be used to identify candidate treatment zones. For example, the candidate treatment zones may be identified as corresponding to image frames that are not within an identified lesion, are a minimum distance away from an identified lesion, have a plaque burden less than a lesion burden, have a visible EEL greater than or equal to an arc threshold, or the like.

One aspect of the disclosure is directed to a method, comprising receiving, by one or more processors, vessel data comprising intravascular imaging data, identifying, by the one or more processors based on the vessel data, a region of interest of a vessel, identifying, by the one or more processors, a first subset of frames within the region of interest having a plaque burden below a threshold plaque burden, the first subset of frames including a threshold number of frames, determining, by the one or more processors, an external elastic lamina (“EEL”) value for the first subset of frames, determining, by the one or more processors based on the EEL values of the first subset of frames, a representative EEL value for the region of interest, and determining, by the one or more processors based on the representative EEL value, the plaque burden for a second subset of frames within the region of interest, wherein the second subset of frames is different than the first subset of frames.

The region of interest may be defined between a first vessel branch and a second vessel branch. The EEL value may be an EEL diameter or an EEL area, and the representative EEL value may be a representative EEL diameter or a representative EEL area. The threshold plaque burden may correspond to a plaque burden at or about 0-50%. The threshold number of frames may be at least three frames.

Determining the plaque burden may be further based on a lumen area for a respective frame. Determining the plaque burden for the respective frame may comprise calculating as 1−(the lumen area/the representative EEL value).

The method may further comprise identifying, by the one or more processors based on the determined plaque burden, lesions. The method may further comprise identifying, by the one or more processors based on the identified lesions, a candidate treatment zone. Identifying the candidate treatment zone may further comprise identifying, by the one or more processors, frames proximal and distal to the identified lesions, wherein at least one of the plaque burden of the frames proximal and distal to the identified lesions is less than a threshold or a visible EEL arc is greater than or equal to an arc threshold.

When a first lesion is within a minimum gap distance from a second lesion, the method further may comprise identifying, by the one or more processors, the first lesion and the second lesion as a combined lesion.

Another aspect of the disclosure is directed to a system comprising one or more processors. The one or more processors may be configured to receive vessel data comprising intravascular imaging data, identify, based on the vessel data, a region of interest of a vessel, identify a first subset of frames within the region of interest having a plaque burden below a threshold plaque burden, the first subset of frames including a threshold number of frames, determine an external elastic lamina (“EEL”) value for the first subset of frames, determine, based on the EEL values of the first subset of frames, a representative EEL value for the region of interest, and determine, based on the representative EEL value, a plaque burden for a second subset of frames within the region of interest, wherein the second subset of frames is different than the first subset of frames.

Yet another aspect of the disclosure is directed to a -transitory computer-readable medium storing instructions, which when executed by one or more processors, cause the one or more processors to receive vessel data comprising intravascular imaging data, identify, based on the vessel data, a region of interest of a vessel, identify a first subset of frames within the region of interest having a plaque burden below a threshold plaque burden, the first subset of frames including a threshold number of frames, determine an external elastic lamina (“EEL”) value for the first subset of frames, determine, based on the EEL values of the first subset of frames, a representative EEL value for the region of interest, and determine, based on the representative EEL value, a plaque burden for a second subset of frames within the region of interest, wherein the second subset of frames is different than the first subset of frames.

One aspect of the disclosure is directed to a method, comprising receiving, by one or more processors, vessel data comprising intravascular imaging data, identifying, by the one or more processors based on the vessel data, a region of interest of a vessel, determining, by the one or more processors based on the vessel data, a predicted elastic external lamina (“EEL”) value for frames within the region of interest as a function of intima thickness and media thickness, and determining, by the one or more processors based on the predicted EEL value, a plaque burden for frames within the region of interest.

The region of interest may be defined between a first vessel branch and a second vessel branch. The predicted EEL value may be further determined as a function of at least one of a lumen area or a lumen diameter derived from a H-K model.

Determining the plaque burden may be further based on a lumen area for a respective frame. Determining the plaque burden for the respective frame may include calculating 1−(the lumen area/the predicted EEL value).

The method may further comprise identifying, by the one or more processors based on the determined plaque burden, lesions. The method may further comprise identifying, by the one or more processors based on the identified lesions, a candidate treatment zone. Identifying the candidate treatment zone may further comprise identifying, by the one or more processors, frames proximal and distal to the identified lesions, wherein at least one of the plaque burden of the frames proximal and distal to the identified lesions is less than a threshold or a visible EEL arc is greater than or equal to an arc threshold. When a first lesion is within a minimum gap distance from a second lesion, the method may further comprise identifying, by the one or more processors, the first lesion and the second lesion as a combined lesion.

The method may further comprise comparing, by the one or more processors, the predicted EEL value for the region of interest to a representative EEL value for a proximal segment of the vessel, and when the representative EEL value for the proximal segment is less than the predicted EEL value for the region of interest, determining the plaque burden for the region of interest is based on the representative EEL value of the proximal segment.

The method may further comprise comparing, by the one or more processors, the predicted EEL value for the region of interest to a representative EEL value for a distal segment of the vessel, and when the representative EEL value for the distal segment is greater than the predicted EEL value for the region of interest, determining the plaque burden for the region of interest is based on the representative EEL value of the distal segment.

Another aspect of the disclosure is directed to a system, comprising one or more processors. The one or more processors may be configured to receive vessel data comprising intravascular imaging data, identify, based on the vessel data, a region of interest of a vessel, determine, based on the vessel data, a predicted elastic external lamina (“EEL”) value for frames within the region of interest as a function of intima thickness and media thickness, and determine, based on the predicted EEL value, a plaque burden for frames within the region of interest.

Yet another aspect of the disclosure is directed to a -transitory computer-readable medium storing instructions, which when executed by one or more processors, cause the one or more processors to receive vessel data comprising intravascular imaging data, identify, based on the vessel data, a region of interest of a vessel, determine, based on the vessel data, a predicted elastic external lamina (“EEL”) value for frames within the region of interest as a function of intima thickness and media thickness, and determine, based on the predicted EEL value, a plaque burden for frames within the region of interest.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an example system according to aspects of the disclosure.

FIG. 2A is an example intravascular image according to aspects of the disclosure, which may be captured using the system of FIG. 1, according to aspects of the disclosure.

FIG. 2B is an example annotated intravascular image, which may be captured and/or annotated using the system of FIG. 1, according to aspects of the disclosure.

FIG. 2C is an example longitudinal representation, which may be generated using the system of FIG. 1, according to aspects of the disclosure.

FIG. 3 is an example user interface, which may be implemented using the system of FIG. 1, according to aspects of the disclosure.

FIG. 4 is another example user interface which may be implemented using the system of FIG. 1, according to aspects of the disclosure.

FIG. 5 is an example longitudinal representation for identifying lesions which may be generated using the system of FIG. 1, according to aspects of the disclosure.

FIG. 6 is an example user interface identifying candidate treatment zones, which may be implemented using the system of FIG. 1, according to aspects of the disclosure.

FIG. 7 is an example user interface for stent planning which may be implemented using the system of FIG. 1, according to aspects of the disclosure.

FIG. 8 is a flow diagram illustrating a method of determining a plaque burden based on an average EEL value, which can be used in providing the interface screens of FIGS. 3, 4, 6, and 7, according to aspects of the disclosure.

FIG. 9 is a flow diagram illustrating a method of determining a plaque burden based on a predicted EEL value, which can be used in providing the interface screens of FIGS. 3, 4, 6, and 7, according to aspects of the disclosure.

### DETAILED DESCRIPTION

The technology is generally directed to automatically identifying lesions based on the plaque burden within a blood vessel. The plaque burden may be determined as a function of lumen values and external elastic lamina (“EEL”) values. The EEL values may be, for example, the EEL diameter and/or the EEL area. The lumen values and EEL values may be automatically determined based on intravascular image data obtained during pullback of an intravascular probe. In some examples, plaque may have overgrown the vessel such that the EEL values cannot be determined based on the image data. In some examples, the plaque may prevent the EEL from being visible in the intravascular image data to a user or the EEL value may have been erroneously determined. The user may be, for example, an analyst, physician, or the like. In examples where the EEL values cannot be determined based on the image data obtained during the pullback, the EEL values may be predicted.

For a region of interest, such as a region of the vessel between two side branches, the EEL values may be predicted as a representative EEL value. According to some examples, the representative EEL values may be predicted as an average, median, mean, or mode EEL value. For clarity throughout the disclosure, representative EEL values will be used. However, representative EEL may be replaced with median EEL, average EEL and/or mean EEL and, therefore, the use of “representative” is not intended to be limiting.

The representative EEL value may be determined based on a threshold number of frames within the region of interest. For example, the region of interest may have a first set of frames in which the plaque burden of the image frames is below a threshold plaque burden. The threshold plaque burden may be about 0-50%. The first set of image frames may have a threshold number of image frames, such as at least three image frames. The region of interest may have a second set of image frames in which the plaque burden is greater than the threshold plaque burden, the EEL value was not able to be automatically determined, or the like. A representative EEL value may be determined based on the EEL values for the first set of frames. The representative EEL value may be used as the predicted EEL value for the frames in the second set of frames. According to some examples, the representative EEL value may be used as the EEL value for at least one of the frames in the first set of frames. For example, the first set of frames may include one or more frames with erroneously measured EEL values. The representative EEL value may be used for the frames in the first set of frames having an EEL value outside a standard deviation of the representative EEL value, that is outside a predetermined range of the representative EEL value, or the like.

According to some examples, the EEL value may be predicted as a representative EEL value of the threshold number of frames based on the H-K model. For example, when applying a H-K model, the H-K model works under the assumption that the vessel size, e.g., diameter, area, etc., remains substantially constant between two side branches.

In examples where the region of interest does not include a threshold number of frames having a plaque burden less than a threshold plaque burden or visible EEL, the EEL value for the frames in the region of interest may be predicted as a function of intima thickness and media thickness. The intima thickness may be, for example, a minimum intima thickness and the media thickness may be, for example, a minimum media thickness. The minimum intima thickness and minimum media thickness may be determined based on histologic observations of vessels. For example, based on histologic observations of vessel, an intima thickness and media thickness may be determined with a standard deviation. The minimum intima thickness and minimum media thickness may be determined based on the low end of the standard deviation. For example, if the total intima and media thickness is determined to be 427 μm±190 μm, the minimum total intima and media thickness would be 237 μm. According to some examples, the predicted EEL value may be further determined as a function of a lumen area/diameter derived from the H-K model.

The H-K model assumes that the vessel naturally tapers from a proximal segment of the vessel to a distal segment of the vessel. In such an example, the predicted EEL value for the region of interest may be compared to a representative EEL value of a segment proximal segment to the region of interest. According to some examples, the proximal segment may be adjacent to the region of interest or further proximal to the region of interest. If the representative EEL value of the proximal segment is less than the predicted EEL value, the representative EEL value may be used as the predicted EEL value for the region of interest. In some examples, the predicted EEL value for the region of interest may be compared to a representative EEL value of a segment of the vessel distal to the region of interest. In some examples, the distal segment may be adjacent to the region of interest or further distal to the region of interest. If the representative EEL value of the distal segment is greater than the predicted EEL value for the region of interest, the representative EEL value of the distal segment may be used as the predicted EEL value for the region of interest.

The representative EEL value and/or predicted EEL value, may be used to determine the plaque burden for the respective frames in the region of interest. For example, the plaque burden may be determined based on the EEL value, either the median or predicted EEL value, and the lumen area for the respective frame. The median or predicted EEL value may be, for example, the median or predicted EEL area. In some examples, the median or predicted EEL value may be the median or predicted EEL diameter. According to some examples, the plaque burden may be determined using the equation: 1−(lumen area/EEL value). The EEL value may be the average EEL value or the predicted EEL value.

The plaque burden may be used to identify lesions. For example, an image frame having the greatest amount of plaque burden may be identified. According to some examples, the identified image frame may be identified as an image frame exceeding a disease threshold. The disease threshold may be, for example, a percent stenosis, a plaque burden, or the like. In some examples, the disease threshold may be, for example, a plaque burden having a range between about 60-100%. According to some examples, the disease threshold may be a plaque burden of about 70%. In some examples, the disease threshold may be determined based on the disease feature, e.g. plaque burden, percent stenosis, etc., such that the percentage may be more or less than 70% or have a range within or outside of the 60-100% range.

The identified image frame may be the initial image frame for a given lesion. To determine the size and location of the lesion, the plaque burden of frames proximal and distal to the initial frame may be compared to a treatment zone threshold until an image frame proximal and distal to the initial frame has a plaque burden less than the treatment zone threshold. The treatment zone threshold may a plaque burden of about 50%. The treatment zone threshold may, in some examples, be more or less than a plaque burden of 50%. For example, the treatment zone threshold may correspond to a plaque burden of about 45%. In some examples, the treatment zone threshold may be a range, such as a plaque burden of about 0-50%. In some examples, the treatment zone threshold may be patient specific, determined by the physician, etc. In some examples, treatment zone end frame may correspond to a visible EEL arc being greater than or equal to an arc threshold. The arc threshold may be at least or about 180 degrees. Accordingly, the example treatment zone thresholds discussed herein are not intended to be limiting. The image frames between the identified proximal and distal frame are then marked as corresponding to a lesion.

According to some examples, the process of identifying lesions is repeated until all lesions within the vessel are identified. For example, excluding the image frames from the identified lesion, the image frame having the next greatest plaque burden may be identified. The lesion, including the image frame having the next greatest plaque burden, is determined by comparing the plaque burden of frames proximal and distal to the image frame having the next greatest plaque burden to the treatment zone threshold. The plaque burden of frames proximal and distal to the image frame having the next greatest plaque burden may be compared to a treatment zone threshold until an image frame proximal and distal to the initial frame has a plaque burden less than the treatment zone threshold. The image frames between the identified proximal and distal frame are then marked as corresponding to a lesion.

After each lesion is identified, the gap, or distance, between lesions may be determined. In examples where the gap is substantially equal to or less than a threshold distance, the lesions may be combined and identified as a single lesion. For example, when a first lesion is within a minimum gap distance from a second lesion, e.g., the gap between the first and second lesion is less than the threshold distance, the first and second lesion may be identified as a combined lesion. In examples where the gap is greater than the threshold distance, the lesions may be identified as individual lesions. According to some examples, suggested, or candidate, treatment zone may be identified. The treatment zone may be, for example, a stent landing zone, a zone for a balloon angioplasty or laser atherectomy, or the like. In examples where the treatment zone is a stent landing zone, the stent landing zone for a stent may be identified based on the identified lesions.

Predicting EEL values for image frames in which the EEL value could not be determined correctly during the pullback allows for the plaque burden of a given frame to be determined. For example, by predicting the EEL value for an image frame, the plaque burden for each image frame obtained during a pullback may be determined. This allows for more accurate and complete vessel information, as compared to disregarding the image frames where EEL could not be determined during a pullback. Further, by determining the plaque burden for the image frames along the pullback, the detected EEL values and the predicted EEL values may be used to more accurately identifying lesion locations, lengths, severity, and the like. Greater accuracy in identifying lesions may allow for more accurate identification of candidate treatment zones. For example, the plaque burden and lesion locations may be used to determine candidate treatment zone end frames that do not include a lesion and/or have a plaque burden less than a threshold burden. This mitigates the need for user interaction to identify and/or adjust treatment zones to a location outside of a lesion or in a location with a plaque burden less than a threshold. According to some examples, the treatment zone may be a stent landing zone. In some examples, the treatment zone may be for a balloon angioplasty or the like.

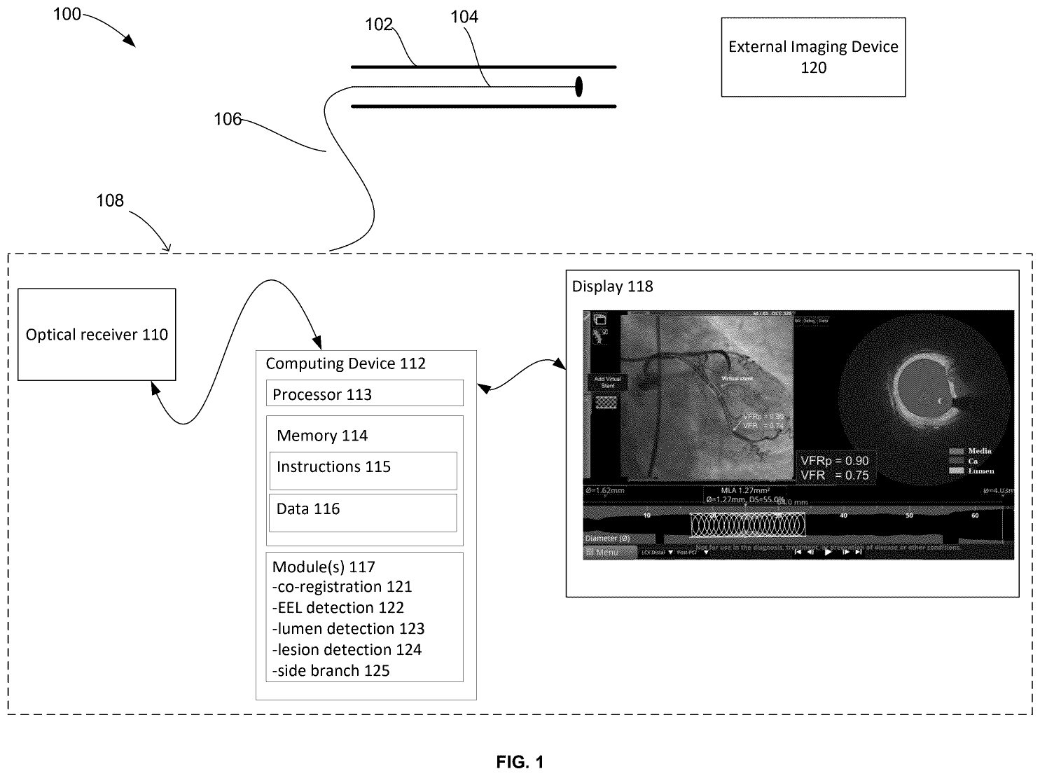

FIG. 1 illustrates a data collection system 100 for use in collecting intravascular and extravascular data. The system may include an intravascular data collection probe 104, external imaging device 120, and subsystem 108. The subsystem 108 may include an optical receiver 110, computing device 112, and display 118.

The data collection probe 104 may be, for example, an optical coherence tomography (“OCT”) probe, an intravascular ultrasound (“IVUS”) catheter, micro-OCT probe, near infrared spectroscopy (NIRS) sensor, optical frequency domain imaging (“OFDI”), or any other device that can be used to image a blood vessel 102. In some examples, the data collection probe 104 may be a pressure wire, a flow meter, etc. The probe 104 may include a probe tip, one or more radiopaque markers, an optical fiber, and a torque wire. Additionally, the probe tip includes one or more data collecting subsystems such as an optical beam director, an acoustic beam director, a pressure detector sensor, other transducers or detectors, and combinations of the foregoing.

While the examples provided herein refer to an intravascular imaging device, such as an OCT probe, the use of an OCT probe is not intended to be limiting. For example, an IVUS catheter, a pressure wire, or another intravascular data collection device may be used in conjunction with or instead of the OCT probe. In general, the disclosure can apply to any intravascular data collection devices can be used to generate and receive signals that include measurement information, such as image data, relative to the blood vessel in which they are used. These devices can include without limitation imaging devices, such as optical or ultrasound probes, pressure sensor devices, and other devices suitable for collecting data with regard to a blood vessel or other components of a cardiovascular system.

A guidewire, not shown, may be used to introduce the probe 104 into the blood vessel 102. The probe 104 may be introduced and pulled back along a length of a blood vessel while collecting data.

The probe 104 may be connected to a subsystem 108 via an optical fiber 106. The subsystem 108 may include a light source, such as a laser, an interferometer having a sample arm and a reference arm, various optical paths, a clock generator, photodiodes, and other OCT, IVUS, micro-OCT, NIRS, and/or pressure wire components.

The probe 104 may be connected to an optical receiver 110. According to some examples, the optical receiver 110 may be a balanced photodiode based system. The optical receiver 110 may be configured to receive light collected by the probe 104. The probe 104 may be coupled to the optical receiver 110 via a wired or wireless connection.

The system 100 may further include, or be configured to receive data from, an external imaging device 120. The external imaging device may be, for example, an imaging system based on angiography, fluoroscopy, x-ray-, nuclear magnetic resonance, computer aided tomography, etc. The external imaging device 120 may be configured to noninvasively image the blood vessel 102. According to some examples, the external imaging device 120 may obtain one or more images before, during, and/or after a pullback of the data collection probe 104. External imaging device 120 may be used to image a patient such that clinical decisions can be made and various possible treatment options such as stent placement can be carried out. These and other imaging systems can be used to image a patient externally or internally to obtain raw data, which can include various types of image data.

The external imaging device 120 may be in communication with subsystem 108. According to some examples, the external imaging device 120 may be wirelessly coupled to subsystem 108 via a communications interface, such as Wi-Fi or Bluetooth. In some examples, the external imaging device 120 may be in communication with subsystem 108 via a wire, such as an optical fiber. In yet another example, external imaging device 120 may be indirectly communicatively coupled to subsystem 108 or computing device 112. For example, the external imaging device 120 may be coupled to a separate computing device (not shown) that is in communication with computing device 112. As another example, image data from the external imaging device 120 may be transferred to the computing device 112 using a computer-readable storage medium.

The subsystem 108 may include a computing device 112. One or more steps may be performed automatically or without user input to navigate images, input information, select and/or interact with an input, etc. In some examples, one or more steps may be performed based on receiving a user input by mouse clicks, a keyboard, touch screen, verbal commands, etc. The computing device 112 may include one or more processors 113, memory 114, instructions 115, data 116, and one or more modules 117.

The one or more processors 113 may be any conventional processors, such as commercially available microprocessors. Alternatively, the one or more processors may be a dedicated device such as an application specific integrated circuit (ASIC) or other hardware-based processor. Although FIG. 1 functionally illustrates the processor, memory, and other elements of device 110 as being within the same block, it will be understood by those of ordinary skill in the art that the processor, computing device, or memory may actually include multiple processors, computing devices, or memories that may or may not be stored within the same physical housing. Similarly, the memory may be a hard drive or other storage media located in a housing different from that of device 112. Accordingly, references to a processor or computing device will be understood to include references to a collection of processors or computing devices or memories that may or may not operate in parallel.

Memory 114 may store information that is accessible by the processors, including instructions 115 that may be executed by the processors 113, and data 116. The memory 114 may be a type of memory operative to store information accessible by the processors 113, including a non-transitory computer-readable medium, or other medium that stores data that may be read with the aid of an electronic device, such as a hard-drive, memory card, read-only memory (“ROM”), random access memory (“RAM”), optical disks, as well as other write-capable and read-only memories. The subject matter disclosed herein may include different combinations of the foregoing, whereby different portions of the instructions 115 and data 116 are stored on different types of media.

Memory 114 may be retrieved, stored or modified by processors 113 in accordance with the instructions 115. For instance, although the present disclosure is not limited by a particular data structure, the data 116 may be stored in computer registers, in a relational database as a table having a plurality of different fields and records, XML documents, or flat files. The data 116 may also be formatted in a computer-readable format such as, but not limited to, binary values, ASCII or Unicode. By further way of example only, the data 116 may be stored as bitmaps comprised of pixels that are stored in compressed or uncompressed, or various image formats (e.g., JPEG), vector-based formats (e.g., SVG) or computer instructions for drawing graphics. Moreover, the data 116 may comprise information sufficient to identify the relevant information, such as numbers, descriptive text, proprietary codes, pointers, references to data stored in other memories (including other network locations) or information that is used by a function to calculate the relevant data.

The instructions 115 can be any set of instructions to be executed directly, such as machine code, or indirectly, such as scripts, by the processor 113. In that regard, the terms “instructions,” “application,” “steps,” and “programs” can be used interchangeably herein. The instructions can be stored in object code format for direct processing by the processor, or in any other computing device language including scripts or collections of independent source code modules that are interpreted on demand or compiled in advance. Functions, methods and routines of the instructions are explained in more detail below.

According to some examples, the computing device 112 may include receive, either by a wired connection or via a wireless connection, data from the probe 104. The data may include, for example, intravascular data including intravascular imaging data, pressure data, temperature data, flow data, or the like. The data received by the computer device 112 from the probe 104 may be used to determine plaque burden, fractional flow reserve (“FFR”) measurements at one or more locations along the vessel, calcium angles, EEL detections, calcium detections, proximal frames, distal frames, EEL-based metrics, stent/no stent decisions, scores, recommendations for debulking and other procedures, evidence based recommendations informed by automatic detection of regions/features of interest, stent planning, etc.

The data obtained by probe 104 and/or external imaging device 120 may processed by one or more modules to provide information about the blood vessel including lumen contours, vessel diameters, vessel cross-sectional areas, lumen area, EEL values, EEL diameters, EEL arcs, lesion locations, lesion size, plaque burdens, VFR, FFR, landing zones, treatment zones, virtual stents bounded by the landing zones or the like. According to some examples, the modules 117 may include an EEL detection module 122, a lumen detection module 123, a lesion detection module 124, and a co-registration module.

The EEL detection module 122 may automatically detect and measure the EEL diameter of a given intravascular image frame taken during a pullback of a vessel. The lumen detection module 123 may automatically detect and measure the lumen diameter of a given intravascular image frame taken during a pullback of the vessel. The lesion detection module 124 may automatically detect lesions within the vessel based on vessel data obtained from the probe 104 and/or external imaging device 120. The side branch detection module 125 may process the vessel data obtained from the probe 104 and/or external imaging device 120 to detect one or more side branches of the blood vessel.

The computing device 112 may be adapted to co-register vessel data obtained during a pullback of the probe 104 with intravascular image and/or an extraluminal image. For example, the computing device 112 may be configured to receive and store extraluminal image data, such as image data generated by external imaging device 120 and obtained by a frame grabber. The computing device 112 may be configured to receive and store intravascular image data, such as image data generated by probe 104 and obtained by the frame grabber. In some examples, computing device 112 may access co-registration module 121 to co-register the vessel data with the luminal image. The luminal image may be an extraluminal image, such as an angiograph, x-ray, or the like. The co-registration module 121 may co-register intravascular data, such as an intravascular image, plaque burden, EEL measurement, lumen diameter measurements, pressure readings, virtual flow reserve (“VFR”), fractional flow reserve (“FFR”), resting full-cycle ratio (“RFR”), flow rates, etc. with the extraluminal image. In some examples, the co-registration module 121 may co-register intravascular data with an intraluminal image, such as an intraluminal image captured by an OCT probe, IVUS probe, micro-OCT probe, or the link.

In one example, the co-registration module 121 may co-register intraluminal data captured during a pullback with one or more extraluminal images. For example, the extraluminal image frames may be pre-processed. Various matrices such as convolution matrices, Hessians, and others can be applied on a per pixel basis to change the intensity, remove, or otherwise modify a given angiography image frame. As discussed herein, the preprocessing stage may enhance, modify, and/or remove features of the extraluminal images to increase the accuracy, processing speed, success rate, and other properties of subsequent processing stages. A vessel centerline may be determined and/or calculated. In some examples, the vessel centerline may be superimposed or otherwise displayed relative to the pre-processed extraluminal image. According to some examples, the vessel centerline may represent a trajectory of the collection probe 104 through the blood vessel during a pullback. In some examples, the centerline may be referred to as a trace. Additionally or alternatively, marker bands or radiopaque markers may be detected in the extraluminal image frames. According to some examples, the extraluminal image frames and the data received by collection probe 104 may be co-registered based on the determined location of the marker bands.

According to some examples, the modules may additionally or alternatively include a video processing software module, a preprocessing software module, an image file size reduction software module, a catheter removal software module, a shadow removal software module, a vessel enhancement software module, a blob enhancement software module, a Laplacian of Gaussian filter or transform software module, a guidewire detection software module, an anatomic feature detection software module, stationary marker detection software module, a background subtraction module, a Frangi vesselness software module, an image intensity sampling module, a moving marker software detection module, iterative centerline testing software module, a background subtraction software module, a morphological close operation software module, a feature tracking software module, a catheter detection software module, a bottom hat filter software module, a path detection software module, a Dijkstra software module, a Viterbi software module, fast marching method based software modules, a vessel centerline generation software module, a vessel centerline tracking module software module, a Hessian software module, an intensity sampling software module, a superposition of image intensity software module and other suitable software modules as described herein. According to some examples, the modules may include software such as preprocessing software, transforms, matrices, and other software-based components that are used to process image data or respond to patient triggers to facilitate co-registration of different types of image data by other software-based components or to otherwise perform annotation of image data to generate ground truths and other software, modules, and functions suitable for implementing various features of the disclosure. The modules can include lumen detection using a scan line based or image based approach, stent detection using a scan line based or image based approach, indicator generation, apposition bar generation for stent planning, guidewire shadow indicator to prevent confusion with dissention, side branches and missing data, and others.

In some examples, the modules may be configured to process the vessel data obtained by the probe 104 and/or external imaging device 120 using machine learning algorithms, artificial intelligence, or the like.

According to some examples, the computing device 112 may include a module for classifying different features within the vessel, e.g., a classifying module. The module for classifying the different features may be a machine learning system (“MLS”) that is implemented by training a classifier to segment or operate upon an image such that its constituent tissues, tissue types, and other regions of interest are detected and characterized based on type or another parameter. In one example, the lumen, intima, media and plaque are detected and identified as having boundaries corresponding to these different tissues.

Training a given MLS/neural network involves using known inputs and known outputs to teach the network.

The training data may include, for example, ground truth data, along with the image data elements, which may include various image formats, such as raw gray scale images, is provided as input to the convolutional neural network (“CNN”) of the MLS. The MLS is run over a period of epochs until the training error is reduced, minimized, or otherwise below a threshold. In one embodiment, the period ranges from about 100 to about 1000 epochs.

After a given MLS/CNN has been trained, it can then process raw images and output the image segmentation that includes classified or characterized tissue. In one example, a polar image from a pullback of an intravascular imaging probe is operated upon by a trained MLS to generated classified images.

According to some examples, various pre-processing steps/operations may be implemented to further improve training and or processing time.

The pre-processing steps can include lumen detection using a MLS that has been previously trained using a training set with annotated lumen regions or segments. The-pre-processing steps can also be selected to speed training of the network and/or the predictive speed of the trained network during backward propagation. Accordingly, the pre-processing steps can also include image data flattening, a circle shift process, a circular shift process, excluding of portions of image data, such as depth data below a noise floor, data removal can be performed on an alternating basis such that every other scan line of an image is removed or every other column is removed, pixels may be filtered to remove noise and increase uniformity of regions, and other pre-processing steps.

In some examples, pre-processing steps may include a normalizing step. The normalizing step may include normalizing intensity of one or more or all of image data, ground truth annotations, masks, frames, scan lines, neural network outputs, and other intensity-based data such that the intensity is normalized within a range such as from about 0 to about 1, or another applicable range. In some examples, MLS training and prediction is performed on polar images and after classification has occurred, the polar views are converted to Cartesian views and the annotations generated by the trained MLS are displayed on the image using color coding or other suitable indicia or visualizations.

A given cost function may provide a metric to evaluate the output of a machine learning system by comparing the ground truth input/training set with the expected output when operating on patient data. The goodness of fit between the training data and the output data can be measured with a cost function. The output of a cost function can be a value that corresponds to an error metric. This output is a comparative metric such as a difference or a distance to summarize how the machine learning system or the neural network or other operative components thereof is succeeding in terms of accurate predictions given the predicative results and the ground truth used to train the system. If the output result of the cost function where zero, the system would be effectively working perfectly. As such, iterative changes to the system can be used to reduce the cost or error function of the system and improve its predictive accuracy.

In addition, a pixel-wise based cost function is specified to measure the distance or another suitable metric or score between prediction and ground truth. In some examples, backpropagation is to update each of the weights in the network based values derived from the cost function. In one embodiment, on partial derivatives of the cost function are used to update the weights during backpropagation. This weight updating process has the benefit of the actual predictive results being closer the ground truth. This has the benefit of reducing or minimizing the error for each output neuron/node of the neural network.

In some examples, annotated masks regions corresponding to set or group of pixels define a ground truth mask that are used to train one or more neural networks disclosed herein. Once the neural network is trained, predictive or detected masks are generated that include sets of pixels that correspond to regions of user data as well as an identifier of the feature or class of the region, such as whether it is lumen, calcium, EEL, or another class or feature disclosed herein. In one example, predictive results are generated on a per class basis and then all of the predictive results for a given image data input, such as an input frame of OCT, IVUS, or other image data, are compared on a pixel-wise bases to generate a final predictive result for all classes. In one example, the predictive results are displayed as an output image mask with regions corresponding to a particular class so indicated by an indicia such as color and one or more legends summarizing which indicia maps to which class.

According to some examples, the classifying module may classify the type of plaque that is present. For example, the classifying module may classify the plaque as calcified. In addition, given that the present of a plaque and other detectable features of a given section of an artery can indicate the presence of a constriction such as from a stenosis, another feature of the disclosure is the ability to quickly and automatically obtain one or more scores associated with a given plaque or stenosis to help facilitate decision making by an end user. For example, a given score determined using the image data and the machine learning-based analysis thereof can help determine whether no immediate action is recommended, or if a stent should be placed relative to a stenosis, or if an artherectomy or other procedure such as bypass is warranted.

For a healthy patient, arteries have various layers arranged in a consistent structure that include the intima, media and adventitia. As a result of the process of atherosclerosis, the intima becomes pathologically thickened and may contain plaques composed of different types of tissues, including fiber, proteoglycans, lipid and calcium, as well as macrophages and other inflammatory cells. These tissue types have different characteristics when imaged using various imaging systems that can be used to establish a set of training data for one or more of the machine learning systems of the disclosure. The plaques that are believed to be most pathologically significant are the so-called vulnerable plaques that consist of a fibrous cap with an underlying lipid pool. Different atherosclerosis plaques have different geometrical shapes. For examples, the foam cells usually form ribbon-like features on the shoulders of large lipid pool; the media appears like annulus around the vessel, etc. The shape information is currently used in qualitative assessment of OCT images. In one example, the neural net is trained to identify fibrous cap and/or fibrous cap with an underlying lipid pool. In various examples, references to calcium herein also include calcified plaques and other calcium containing tissue, without limitation.

The ability to quickly perform an imaging procedure on a patient and obtain arterial images and then processes the images using a machine learning system while the patient is still catheterized and prepared to receive a stent or other treatment option results in significant time savings and improvements in patient outcomes. The classifying module may be similar to the classification methods described in U.S. Pat. No. 11,250,294 entitled “SYSTEMS AND METHODS FOR CLASSIFICATION OF ARTERIAL IMAGE REGIONS AND FEATURES THEREOF,” the contents of which are incorporated by reference herein in their entirety.

The subsystem 108 may include a display 118 for outputting content to a user. The display 118 may be integrated with the computing device 112, or it may be a standalone unit electronically coupled to the computing device 112. The display 118 may output intravascular data relating to one or more features detected in the blood vessel and/or obtained during a pullback. For example, the output may include, without limitation, cross-sectional scan data, longitudinal scans, three-dimensional representations generated based on intraluminal and/or extraluminal images, diameter graphs, image masks, lumen border, plaque sizes, plaque circumference, visual indicia of plaque location, visual indicia of risk posed to stent expansion, flow rate, suggested treatment zones, or the like. The display 118 may identify features with text, arrows, color
coding, highlighting, contour lines, or other suitable human or machine-readable indicia.

According to some examples the display 118 may include a graphic user interface (“GUI”). The display 118 may be a touchscreen display in which a user can provide an input to navigate images, input information, select and/or interact with an input, etc. In some examples, the display 118 and/or computing device 112 may include an input device, such as a trackpad, mouse, keyboard, etc. that allows a user to navigate images, input information, select and/or interact with an input, etc. The display 118 alone or in combination with computing device 112 may allow for toggling between one or more viewing modes in response to user inputs. For example, a user may be able to toggle between different intravascular data, images, etc. recorded during each of the pullbacks.

In some examples, the display 118, alone or in combination with computing device 112, may present one or more menus as output to the physician, and the physician may provide input in response by selecting an item from the one or more menus. For example, the menu may allow a user to show or hide various features. As another example, there may be a menu for selecting blood vessel features to display.

The output may include image data, such as cross-sectional images obtained by the probe 104 during a pullback and/or external images obtained by the external imaging device 120. In some examples, the output may include a longitudinal representation of the vessel, a graphical representation of the vessel data, a three-dimensional representation of the vessel and/or vessel data, or the like. The representations of the vessel may provide various viewing angles and section views. In some examples, EEL positions, diameters thereof, or other EEL based parameters may be output for display relative to angular measurements, detected calcium arcs, plaque burden, or the like.

In some examples, one or more visual representations of the images may include an indication of a lesion location, lesion severity, lesion length, or the like. Additionally or alternatively, the indication of the lesion may be color coded, where each color represents the severity, length, or other measurement related to the lesion.

In some examples, the output may include candidate treatment zones. The candidate treatment zone may be, for example, a candidate stent landing zone. For example, the output may include an indication corresponding to a candidate proximal landing zone for a stent and a candidate distal landing zone for a stent. The candidate treatment zone may be determined based on the determined plaque burden, lesion locations, lesion length, or the like. The indications may be provided on any of the vessel representations, e.g., the three-dimensional representation, the longitudinal representation, the graphical representation, image data such as the external images, etc.

According to some examples, the display 118 and/or computing device 112 may be configured to receive one or more inputs corresponding to a selection on one or more representations. For example, an input may be received corresponding to a selection of an image frame on the longitudinal representation. In response, the other representations provided for output may be updated to display a corresponding indication or image frame. For example, the extraluminal image may be updated to have an indication along the vessel corresponding to the location of the image frame selected in the longitudinal representation, a circumferential indication may be provided on a three-dimensional representation corresponding to the location of the image frame selected in the longitudinal representation, the cross-sectional image frame may be updated to correspond to the image frame selected in the longitudinal representation, etc. In some examples, the vessel data associated with the selected location may be updated and provided for display.

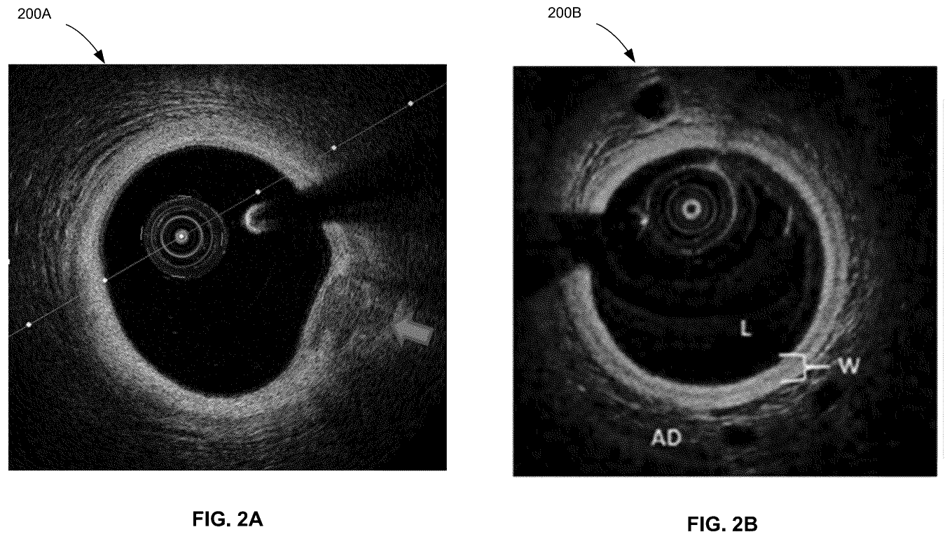

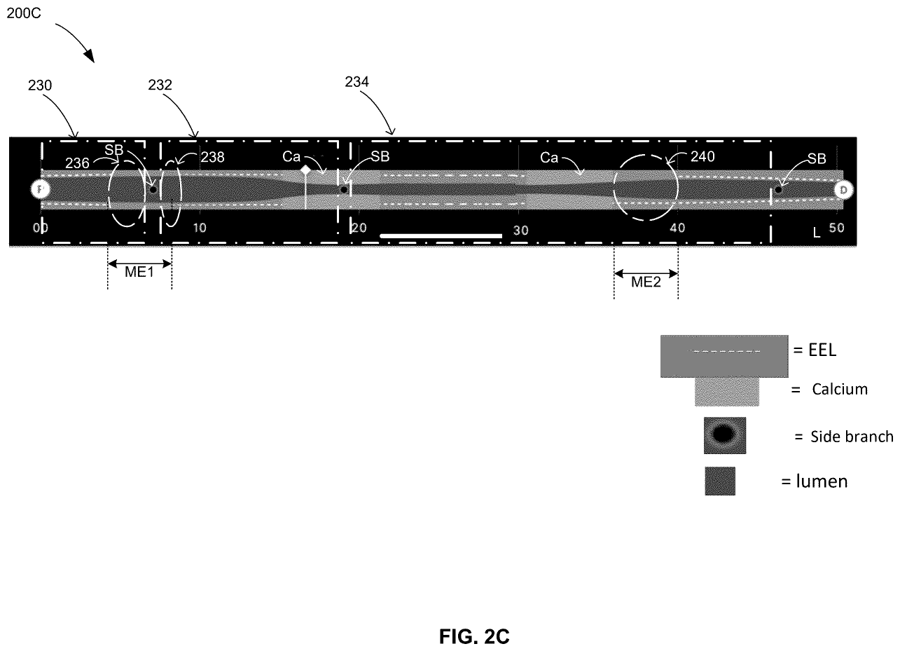

FIG. 2A is an example image frame obtained during a pullback of the probe 104 of FIG. 1. For example, during the pullback of the probe 104, a plurality of image frames may be obtained. The image frame 200A may be an OCT image frame, IVUS image frame, NIRS image frame, OFDI image frame, or the like.

The image frame 200A may be processed using one or more modules 117 to determine vessel data. The vessel data may include, for example, vessel measurements such as EEL measurements, lumen measurements, plaque burden, calcium burden, intima thickness, media thickness, or the like. For example, the EEL detection module 122 may automatically determine an EEL value for the image frames, including image frame 200A. The lumen detection module 123 may automatically determine the lumen area for the image frames, including image frame 200A. The lesion detection module 124 may automatically determine a plaque burden for the image frames, including image frame 200A. The plaque burden for a given image frame may, in some examples, be determined based on the determined EEL value and lumen area for the given image frame.

FIG. 2B is an example annotated image frame. The image frame 200B may be similar to image frame 200A in that image frame 200B may also have been obtained during a pullback of probe 104. The image frame 200B may, therefore, be an OCT image frame, IVUS image frame, MRS image frame, OFDI image frame, or the like.

The image frame 200B may be processed similarly to image frame 200A such that vessel measurements may be determined. In some examples, the image frames, such as image frame 200B, may be processed to identify different features within the image frame 200B. For example, the image frame 200B may be processed to identify the probe 104, the vessel wall W, the adventitia AD, the lumen L, or the like. In some examples, the image frame 200B may be processed to identify EEL, plaques, calcium plaque, calcium angles, or the like. According to some examples, the output 118 may include an indication of the identified vessel wall W, adventitia AD, lumen, L, calcium angles, plaque burdens, etc.

The lumen L may be identified by the lumen detection module 123. In some examples, the lumen L may be automatically identified and used to determine the lumen area of the vessel in the given image frame. The lumen area may be used to determine the plaque burden within the image frame. For example, an EEL value for a given frame can be determined by the EEL detection module 122, the plaque burden for the frame may be determined as: Plaque Burden=1−(Lumen Area/EEL value). In the equation, the EEL value may be, for example, the EEL area or the EEL diameter.

According to some examples, the EEL value may not be able to be determined automatically for the frame, such as when the plaque burden reduces the visibility of the EEL. In another example, the plaque burden may be greater than a threshold value. For example, when the plaque burden is greater than the threshold value, the plaque burden may be considered unhealthy. In some examples, the EEL value may be erroneously measured, such as when the EEL is not visible enough to be measured. In examples where the EEL value cannot be determined or where plaque burden is greater than the threshold value, the EEL value may be predicted in order to determine the plaque burden for a given frame.

FIG. 2C illustrates an example longitudinal representation of a vessel. The longitudinal representation 200C may be generated based on image frames obtained by probe 104 during a pullback, for example by the computing device 112 of FIG. 1. The longitudinal representation 200C may include an indication “P” corresponding to the proximal end of the pullback and an indication “D” corresponding to the distal end of the pullback. According to some examples, the longitudinal representation 200C may include an indication corresponding to the location of a side branch “SB” along the vessel. The longitudinal representation 200C may be color coded, shaded, or include some type of indication between different tissue types within the vessel. For example, regions of calcium “Ca” may be color coded with a first color while regions without calcium may be a different color.

The longitudinal representation 200C may include an indication of regions, or image frames, in which the image frames include EEL value. For example, the longitudinal representation 200C may include a dashed line along the length of the representation indicating frames, or regions, in which an EEL value was measured or detected. According to some examples, the longitudinal representation 200C may include regions in which the EEL value was not measured and/or the plaque burden was above a threshold. The EEL value may not have been measured or measured incorrectly if the EEL was not visible enough to be measured. For example, regions “ME1” and “ME2” may have a plaque burden above the threshold. The absence of the dotted line in regions ME1, ME2 may indicate that plaque may have overgrown those regions ME1, ME2 such that the EEL is not detected and/or not detected at a level that satisfied the threshold to depict the EEL value. The EEL value may be, for example, the EEL area and/or EEL diameter. In some examples, the absence of the dotted line representing EEL regions may indicate that the EEL value for those image frames were not able to determined.

According to some examples, regions of interest may be identified. The regions of interest 230, 232, 234 may be regions of the vessel between side branches SB. The regions of interest 230, 232, 234 may include subregions 236, 238, 240 in which EEL values were not determined and/or were below a threshold value. In such regions 236, 238, 240 the EEL values may be predicted.

According to some examples, the detected, or determined, EEL values within the region of interest 230, 232, 234 may be used to predict the EEL values in the respective subregions 236, 238, 240. For purposes of clarity herein, the disclosure will be directed to region of interest 232 and subregion 238. The methods described herein, however, may also be applied to regions of interest 230, 234 and respective subregions 236, 240. Accordingly, using region of interest 232 and subregion 238 is just one example and is not intended to be limiting.

Region of interest 232 may be located between two side branches SB. One or more modules 117 may be used to process the image frames within region of interest 232 obtained during the pullback. For example, the EEL values, lumen area values, and plaque burden may be automatically determined. As discussed above, the EEL values may not have been able to be determined within subregion 238 and/or the plaque burden reduces the visibility of the EEL such that the EEL values are not suitable for use when determining the plaque burden. In such an example, the EEL values for the image frames in subregion 238 may be predicted.

In some examples, the predicted EEL values may be determined based on the determined EEL values in the remainder of the image frames within region 232. For example, region 232 may comprise a plurality of image frames. A first subset of frames may have a plaque burden less than a threshold plaque burden. The EEL values may be determined for at least a first subset of the image frames. A second subset of image frames, such as those within subregion 238, had a plaque burden above the threshold plaque burden. In some examples, the EEL values for the second subset of frames may have been erroneously measured due to the plaque burden or EEL values that were undetectable due to the plaque burden. In examples where the first subset of image frames comprises a threshold number of frames having a plaque burden below a threshold plaque burden, the EEL values of the threshold number of frames may be determined. A representative EEL value may be determined based on the EEL values of the image frames within the first subset of image frames that have a plaque burden below the threshold plaque burden. The threshold plaque burden may be, for example, between about 0-50%. For example, the threshold plaque burden may be about 40%, 45%, etc. In some examples, the threshold plaque burden may be more or less than about 0-50%. In another example, the threshold plaque burden may be a range, patient specific, determined by the physician, etc.

According to some examples, the representative EEL value of the image frames within the first subset of image frames may be used as the EEL value for image frames in the first subset of image frames having an EEL value that was erroneously measured. For example, some of the image frames in the first set of image frames may have an EEL value that is greater than a standard deviation from the representative EEL value, outside a predetermined range of the representative EEL value, or the like. In such an example, e.g., where the EEL value is erroneously measured, the representative EEL value of the first set of frames may be used as the EEL value for the erroneously measured frames.

The threshold number of frames may be determined based on the number of frames in the region of interest. For example, for a larger region of interest the threshold number of frames may be greater than the threshold number of frames for a smaller region of interest. In some examples, regardless of the size of the region of interest, the threshold number of frames may be three image frames.

The predicted EEL value and the lumen area may be used to determine the plaque burden for the frames in which the EEL value was not automatically determined, had a plaque burden greater than the threshold plaque burden, had an EEL value that was incorrectly measured, etc. In such an example, the plaque burden for a given frame may be determined as 1−(lumen area/predicted EEL value). In this equation, the predicted EEL value may be the predicted EEL area and/or predicted EEL diameter.

According to some examples, the predicted EEL value may be determined as a function of the intima thickness and media thickness. The intima thickness may be, for example, a minimum intima thickness and the media thickness may be, for example, a minimum media thickness. The minimum intima thickness and minimum media thickness may be determined based on typical vessels. For example, typical vessels having an plaque burden value below the threshold value may be used to determine an average value for the minimum intima thickness and minimum media thickness. In one example, the combined minimum intima thickness and minimum media thickness may be 0.237 mm. In some examples, the respective minimum intima thickness and minimum media thickness may be 0.198 mm. The total combined minimum intima and media thickness of 0.237 mm and the respective minimum intima and media thickness of 0.198 mm are just some examples and are not intended to be limiting. For example, the combined thickness may be greater or less than approximately 0.237 mm and the respective thicknesses may be greater or less than approximately 0.198 mm. In some examples, the respective minimum intima and media thicknesses may be the same value, or they may be different values. According to some examples, the minimum intima and media thickness may be based on histologic observations of vessels. For example, based on histologic observations of vessel, an intima thickness and media thickness may be determined with a standard deviation. The minimum intima thickness and minimum media thickness may be determined based on the low end of the standard deviation. For example, if the total intima and media thickness is determined to be 427 μm±190 μm, the minimum total intima and media thickness would be 237 μm. The total intima and media thickness may change based on the size of the dataset studies, patients participating in the study, or the like. Accordingly, while a total combined minimum intima and media thickness of 0.237 mm is used, the total combined minimum intima and media may be any value determined by histologic observations.

The predicted EEL value may be further determined as a function of a H-K model, where the H-K model predicts the expected lumen diameter. An assumption of the H-K model is that the size of the vessel within the region of interest 232, e.g., between the side branches, is substantially constant. For example, the H-K model predicts the lumen diameter based on the assumption that the diameter, circumference, and/or area of the vessel is substantially constant along the region of interest. According to some examples, the H-K model may provide a prediction of what the lumen diameter for the vessel in a given image frame should be if the vessel was healthy. A healthy vessel may be a vessel having a plaque burden below a threshold plaque burden, a lumen diameter above a threshold diameter value, or the like.

According to some examples, the predicted EEL may be determined using the following equation:

$$Predicted EEL value = ( Expected Lumen Diameter ) + 2 * ( Minimum Intima Thickness + Minimum Media Thickness )$$

In the equation, the predicted EEL value may be the predicted EEL area and/or predicted EEL diameter. The expected lumen diameter may be, for example, the lumen diameter determined using the H-K model.

As the EEL is outside of the lumen, additional distance may be added in the form of the minimum intima thickness and minimum media thickness to predict the EEL value. For example, the minimum intima thickness and minimum media thickness may compensate for the additional width between the lumen and the EEL. The addition of the minimum intima thickness and minimum media thickness may be multiplied by two to compensate for the additional width on both sides of the vessel. By adding the double minimum intima thickness and minimum media thickness to the expected lumen diameter, that was predicted by the H-K model, the predicted EEL value may be determined.

The predicted EEL value and the lumen area may be used to determine the plaque burden for the frames in which the EEL value was not automatically determined and/or incorrectly determined. In such an example, the plaque burden for a given frame may be determined as 1−(lumen area/predicted EEL value).

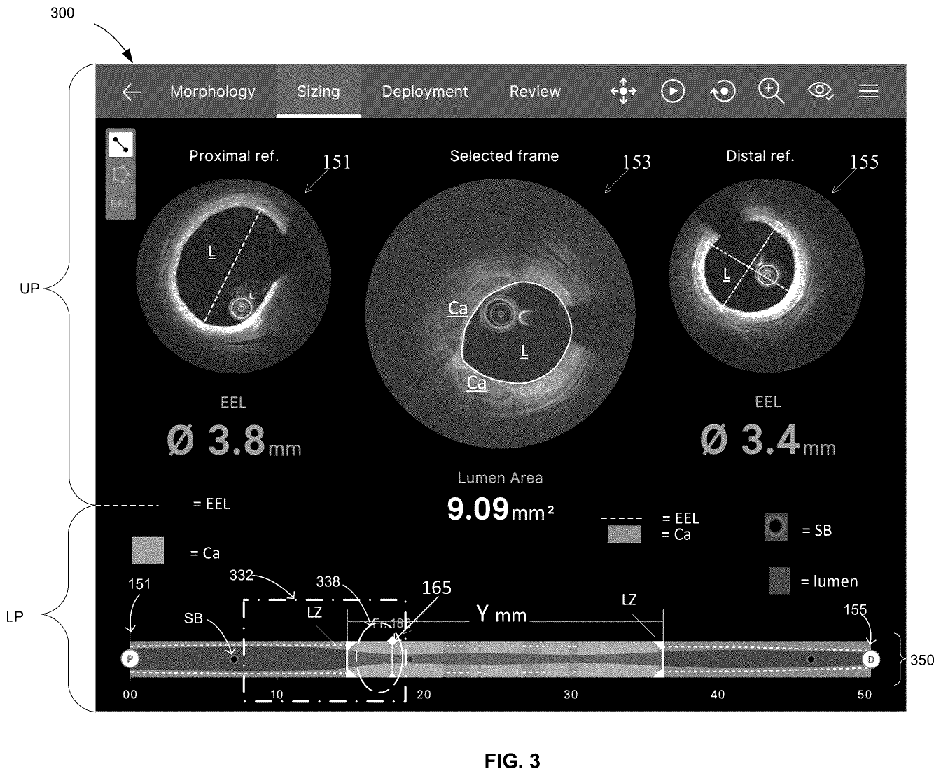

FIG. 3 illustrates an example user interface for use on the display 118 of FIG. 1. The user interface 300 may include an upper portion “UP” of the interface and a lower portion “LP” of the interface. The upper portion UP may include image frames, such as cross-sectional images of the vessel taken during a pullback, and the lower portion LP may include a longitudinal representation 350 of the vessel. The longitudinal representation 350 may be generated using the image frames captured by probe 104 during a pullback.

In the upper portion UP of the interface 300, there may be three image frames corresponding to image frames or particular views or slices of a vessel at proximal reference 151, the selected frame 153 corresponding to the image frame with diamond 165 in the longitudinal representation 350, and a view at distal reference frame 155.

The proximal and distal reference frames 151, 155 illustrate the lumen L of the vessel within the image frame with one or more dotted lines passing through lumen L. These values from measuring these lines may be applied to measured or detected EEL positions, points, or pixels. According to some examples, these values may be used to generate a measured EEL value, such as an EEL diameter, or an average EEL diameter. Some exemplary EEL diameter measure is shown as about 3.8 mm (proximal) and about 3.4 (distal). According to some examples, the measured EEL values may be used to predict EEL values for one or more frames in which the EEL value cannot be determined.

The lower portion LP of the interface 300 may illustrate a combined calcium “Ca” and EEL as the longitudinal representation 350. The longitudinal representation 350 may be similar to the longitudinal representation 200C in that the longitudinal representation 350 may include an indication of one or more side branches SB, EEL values, areas of calcium, or the like. According to some examples, the interface 300 may include an indication of one or more regions of interest 332, such as regions of the vessel located between two side branches. In some examples, the interface 300 may include an indication of subregion 338, in which the EEL values of image frames within the subregion could not be determined. The EEL values of the image frames within subregion 338 may be predicted as a median of EEL values within region 332 when there is a threshold number of image frames having a plaque burden less than a threshold plaque burden and/or a visible EEL arc is greater or equal to an arc threshold. The arc threshold may be, for example, at least or about 180 degrees. The threshold plaque burden may be, for example, a healthy plaque burden. The healthy plaque burden may, in some examples, correspond to a plaque burden between 0-50%. In some examples, the healthy plaque burden may correspond to a plaque burden between 35-50%.

In other examples, the healthy plaque burden may correspond to a range of plaque burden greater than 50%. Accordingly, a plaque burden between 0-50% and 35-50% are just some examples and are not intended to be limiting.

In some examples, the EEL values of the image frames within subregion 338 may be predicted as a function of an H-K model, minimum intima thickness, and minimum media thickness. For example, when there are not enough image frames having a plaque burden below the threshold plaque burden, the EEL value of the image frames may be predicted as a function of the H-K model, minimum intima thickness, and minimum media thickness.

According to some examples, the predicted EEL value, determined as a function of the H-K model, may be compared to an EEL value of a proximal segment of the vessel and/or a distal segment of the vessel. The proximal segment and/or the distal segment of the vessel may be a segment of the vessel adjacent to the region of interest or further proximal and/or distal to the region of interest. For example, there may be a distance between the proximal segment and the region of interest and/or the distal segment and the region of interest. The EEL value of the proximal segment and/or the distal segment of the vessel may be, in some examples, a representative EEL value of the proximal and/or distal segment of the vessel. If the predicted EEL value is less than the representative EEL value of the distal segment, than the representative EEL value of the distal segment may be used to determine the plaque burden of frames within the region of interest. The representative EEL value of the distal segment may be used when the predicted EEL value is less than the representative EEL value of the distal segment due to the H-K model assuming that the vessel has a natural taper from a proximal vessel segment to a distal vessel segment. If the predicted EEL value is greater than the representative EEL value of the proximal segment, the representative EEL value of the proximal segment may be used to determine the plaque burden of the frames within the region of interest. The representative EEL value of the proximal segment may be used when the predicted EEL value is greater than the representative EEL value of the proximal segment due to the H-K model assuming that the vessel has a natural taper from a proximal vessel segment to a distal vessel segment.

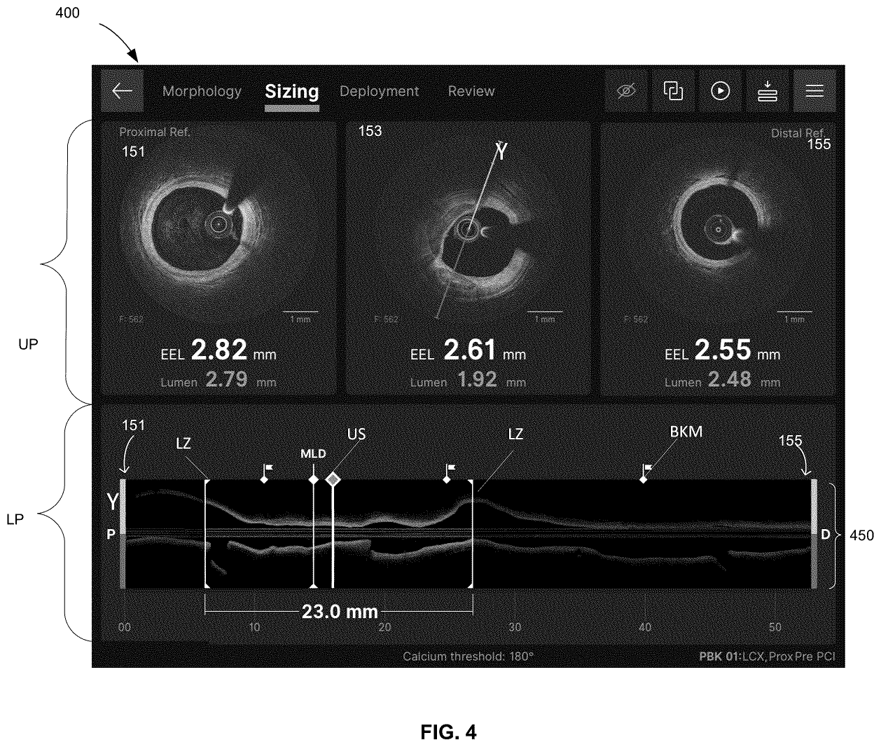

FIG. 4 illustrates another example user interface for use on the display 118 of FIG. 1. User interface 400 may be similar to user interface 300 having an upper portion “UP” and a lower portion “LP.” The difference between interface 300 and interface 400 may be the representations output for display. For example, the longitudinal representation 350 provided for display in interface 300 may be lumen profile whereas the longitudinal representation 450 provided for display in interface 400 may be a lateral representation of the vessel, e.g., an L-view representation.

As shown, the upper portion UP of interface 400 includes three frames, with a user selectable frame in the middle. Upon receiving an input corresponding to the selection and/or movement of marker “US,” the user selected frame 153 may be updated. Additionally, the vessel data associated with the user selected frame 153 may be updated to correspond to the selected frame. This may allow different EEL and lumen diameters to be considered relative to proximal and distal references 151, 155. A bookmark BKM is also shown that can be set by a user using GUI so they can move between frames quickly. Another marker showing the frame with the minimum lumen diameter MLD is shown, in other instances the MLA, or minimum lumen area can be displayed.

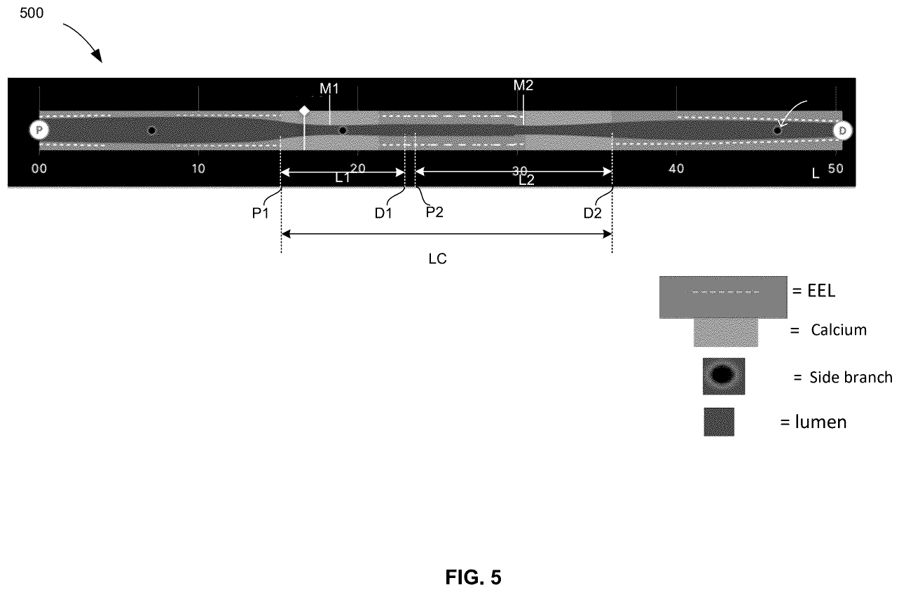

FIG. 5 illustrates an example longitudinal representation of a vessel which can be generated using the computing device 112. According to some examples, the longitudinal representation 500 and/or the data used to generate the longitudinal representation 500 may be used to automatically identify lesions. For example, a lesion may be identified based on the plaque burden for each frame. The plaque burden may be determined as a function of the EEL value and lumen area for a given frame. In some examples, a determined EEL value may be used and, in some examples, a predicted EEL value may be used to determine the plaque burden. The plaque burden may be determined as 1−(lumen area/EEL value), where the EEL value is the determined EEL value or the predicted EEL value for the image frame. Frames in which the plaque burden is above the threshold plaque burden may be marked, or indicated, as being part of a lesion. The threshold plaque burden may be, for example, a plaque burden of 70%. In some examples, the threshold plaque burden may be, for example, a plaque burden of 25%, 50%, etc. The threshold plaque burden may be patient specific. In some examples, the threshold plaque burden may be adjusted by a physician to be correspond to a plaque burden of any value or percentage. The example of 70% is, therefore, just one example and is not intended to be limiting.

As shown in the longitudinal representation 500, two different lesions L1, L2 may identified. According to some examples, based on the proximity of each lesion L1, L2 to each other, the lesions L1, L2 may be identified as a single lesion LC.

To identify lesion L1, an image frame M1 with a plaque burden greater than the threshold plaque burden may be identified. According to some examples, the image frame with a maximum plaque burden may be identified. In some examples, the image frame with a plaque burden greater than a disease threshold may be identified. The plaque burden of each frame in the proximal and distal direction of the identified image frame may be determined and compared to a treatment zone threshold until a proximal image frame P1 and a distal image frame D1 is identified as having a plaque burden less than the treatment zone threshold. The treatment zone threshold may be, for example, a plaque burden of about 0-50%. The treatment zone end frames may also be defined as frames P1, D1 that have a plaque burden less than the treatment zone threshold and/or have a visible arc of EEL greater than or equal to an arc threshold. The arc threshold may be, for example, at least or about 180 degrees. The lesion L1 may be identified as extending between the identified proximal and distal image frames P1, D1.

This process may be repeated until all lesions, e.g., lesions L2, are identified for the pullback. For example, the image frame with the next highest plaque burden from those remaining, e.g., the image frames besides the image frames identified as being lesion L1, may be identified. For example, the next highest plaque burden may be M2. According to some examples, the image frame having a plaque burden greater than the disease threshold may be identified from the remaining image frames. Similar to identifying lesion L1, lesion L2 may be identified by comparing the plaque burden of proximal and distal image frames until identifying proximal image frame P2 and distal image frame D2 having a plaque burden less than the treatment zone threshold and/or having a visible EEL arc greater than or equal to an arc threshold. The arc threshold may be, for example, at least or about 180 degrees. While only two lesions L1, L2 have been identified on the longitudinal representation 500, there may be more lesions or less lesions identified for a given pullback.

According to some examples, after identifying one or more individual lesions L1, L2, the lesions may be combined into a single lesion LC. For example, one or more individual lesions may be combined into a single lesion when there is less than a threshold distance between the individual lesions. The distance between lesions may be measured based on the number of image frames between the distal end of a first lesion and a proximal end of another lesion. In some examples, the distance between lesions may be measured in millimeters or any other form of distance measurement. The threshold distance may be patient specific, updated by the physician, preset, or the like. In some examples, the threshold distance between lesions may be between about 5-10 mm. According to some examples, the threshold distance may be between about 8-10 mm. In other examples, the threshold distance may be less than about 5 mm or greater than about 10 mm. In some examples, the threshold distance may be patient specific, physician preference, or the like. Accordingly, the threshold distance between about 5-10 mm is just one example and is note intended to be limiting.

Using the lesions L1, L2 in FIG. 5 as an example, the distance between distal image frame D1 of lesion L1 and proximal image frame P2 of lesion L2 may be determined. If the distance between distal image frame D1 of lesion L1 and proximal image frame P2 of lesion L2 is less than the threshold distance, the lesions L1 and L2 may be combined. As shown, the distance between lesions L1 and L2 is less than the threshold. Lesions L1, L2 are, therefore, combined into a single lesion LC.

By combining lesions that have a gap, or distance, between them that is less than a threshold distance may prevent a treatment zone end frame from being placed in the gap between lesions. For example, by combining the individual lesions into a single combined lesion, the system may automatically identify a treatment zone end frame to be at a position proximal and distal to the combined lesion, rather than within a gap between two lesions.

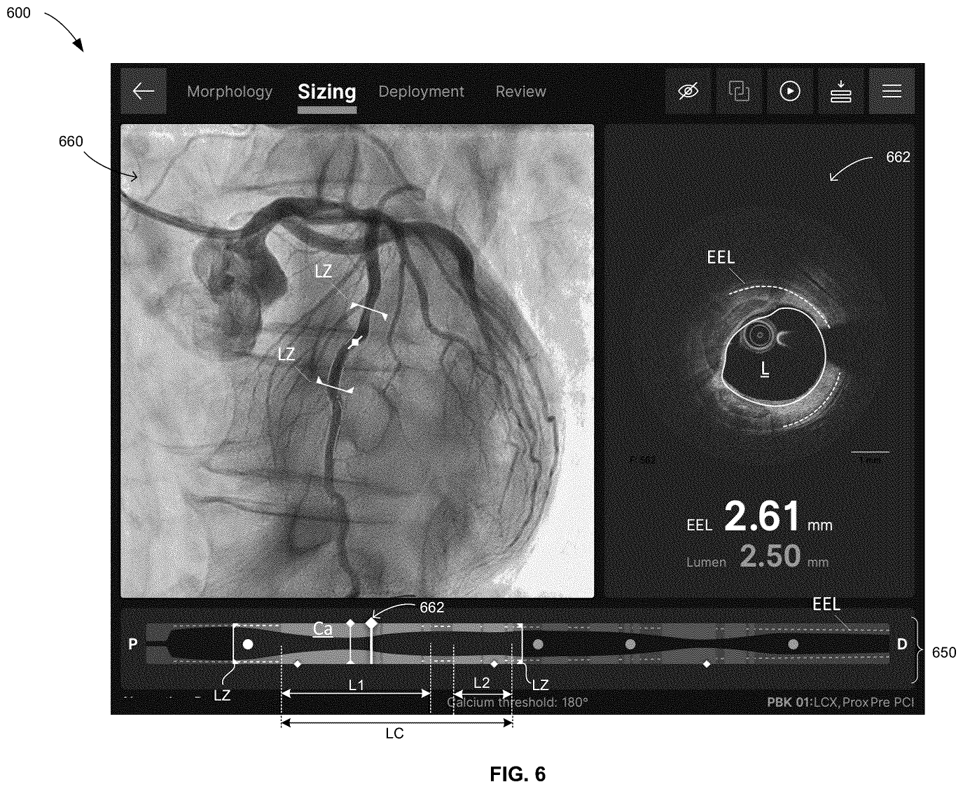

FIG. 6 illustrates another example user interface which may be provided for output on display 118. The user interface 600 illustrates candidate treatment zone end frames “TZ.” The candidate treatment zone end frames TZ may be at a location proximal and distal to the identified lesion LC. For example, the candidate treatment zone end frames TZ for the stent may be determined based on a comparison of the plaque burden of the images frames proximal and distal to the lesion LC to a treatment zone threshold. The treatment zone threshold may be, for example, a plaque burden of about 0-50%. When an image frame proximal and distal to the lesion LC are identified has having a plaque burden that substantially corresponds to or is less than the treatment zone threshold and/or has a visible EEL arc greater than or equal to an arc threshold, the image frame may be identified as a candidate treatment zone end frame for the stent. The arc threshold may be, for example, at least or about 180 degrees. The candidate treatment zones may be provided for output on the longitudinal presentation 650 or any other co-registered representation of the vessel. For example, the indications of the candidate treatment zone end frames TZ may be provided for output on a co-registered external image frame 660, such as an angiography image frame.

Interface 600 may include a cross-sectional representation of the vessel, such as a cross-sectional image frame of the vessel. The cross-section image 662 may include an indication, such as a dashed line, identifying the location of the EEL and an indication, such as a solid line, identifying the lumen “L.” Corresponding vessel data, such as EEL values and lumen diameters values, may be provided for output along with the image frame 662. For example, the cross-sectional image 662 may correspond to the image frame 662 selected on the longitudinal representation 650. As shown, the selected image frame 662, has an EEL diameter of 2.61 mm and a lumen diameter of 2.50 mm. According to some examples, the interface 600 may include an indication of an EEL arc. For example, the interface 600 may output a representation of the EEL arc on the intravascular image, longitudinal representation, or the like. In some examples, the interface 600 may provide an output of the EEL arc as a value, such as a percentage.

These EEL values and lumen values may be used to help inform stent selection or other treatment devices. For example, based on the EEL values and lumen values, a physician may be able to assess the candidate treatment zones TZ that were automatically suggested by the system after. According to some examples, co-registering the longitudinal representation with the extraluminal image such that the candidate treatment zones are displayed on both the longitudinal representation and the extraluminal images assists with assessing and evaluating the candidate treatment zones.

According to some examples, the cross-sectional representation may include an indication of the plaque burden, calcium burden, or the like. For example, the cross-sectional representation may include an arc identifying the regions of plaque burden, calcium burden, etc. In some examples, the indications may be color coded to identify the different classes identified in the cross-sectional representation. The types of classes may include, for example, intima, media, adventitia, lumen, external elastic lamina (EEL), internal elastic lamina (IEL), plaque, calcium, calcium plaques, side branch, guidewire, stent strut, stent, jailed stents, bioresorbable vascular scaffold (BVS), drug eluting stents (DES), fibrous, blooming artifact, pressure wire, lipid, atherosclerotic plaque, stenosis, calcium, calcified plaque, calcium containing tissue, lesions, fat, malapposed stent, underinflated stent, over inflated stent, radio opaque marker, branching angle of arterial tree, calibration element of probe, doped films, light scattering particles, sheath, doped sheath, fiducial registration points, diameter measure, radial measure, guide catheter, shadow region, guidewire segment, length, or thickness.

Additionally or alternatively, the indications may be color coded to correspond to the severity of the plaque burden, lesion, calcium build up, etc. The color coding provided for output on the cross-sectional representation may, in some examples, be provided for output on the longitudinal representation and/or the extraluminal image.

According to some examples, the interface 600 may include a three-dimensional (“3D”) representation of the vessel, including the region of interest. The 3D representation may in addition to or as an alternative of one of the other representations of the vessel. For example, the interface 600 may include one or more of an extra-luminal image, a cross-sectional image frame, a longitudinal representation, a 3D representation, or the like. In some examples, the longitudinal representation may be symmetric about the longitudinal axis of the vessel and/or representation. In another example, the longitudinal representation may be a carpet view of the vessel. Each representation of the vessel, e.g., the extra-luminal image, cross-sectional image, 3D representation, longitudinal representation, etc., may be configured to be color coded, include annotation and/or indications, or the like.

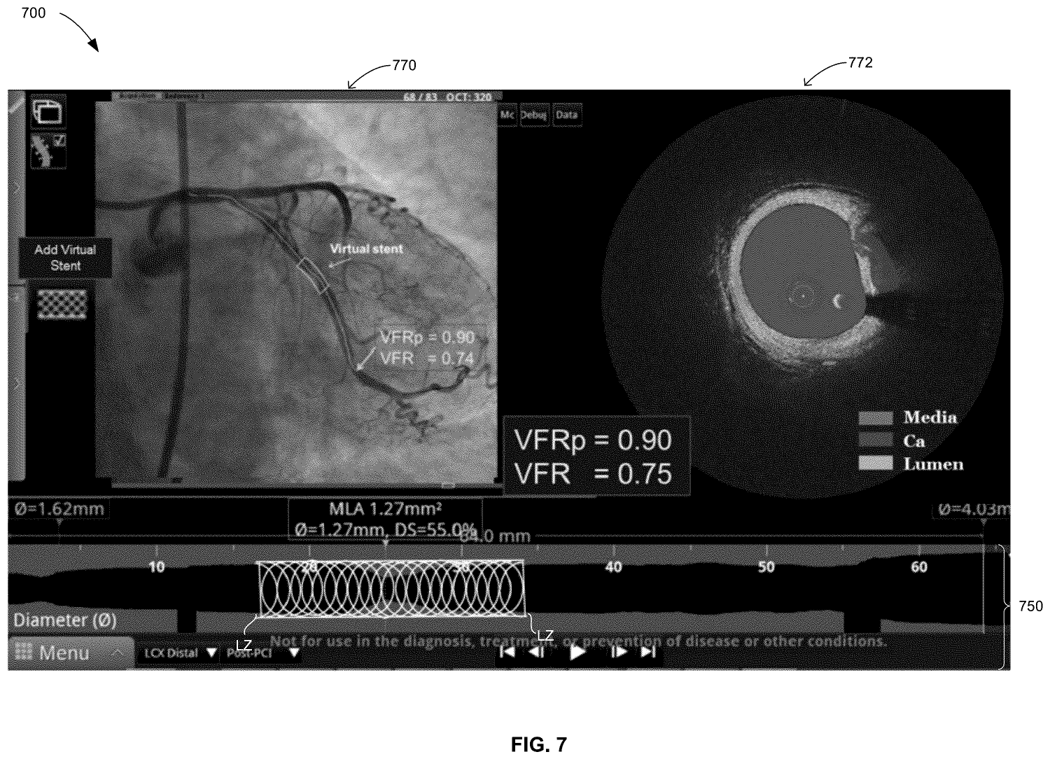

FIG. 7 illustrates another example user interface which may be provided for output on display 118. The user interface 700 may be used to support stent planning through virtual stenting. In some examples, the virtual stenting may correspond to the treatment zone end frames TZ that were automatically identified based on the detected lesions. A depiction, or an indication, of the virtual stent may be provided on one or more of the representations of the vessel. For example, similar to interface 600, interface 700 may include one or more representations of the vessel include an extraluminal image 770, a cross-sectional image 772, and a longitudinal representation 750. An indication of the virtual stent may be provided for output on the extraluminal image 770 and/or the longitudinal representation 750.

According to some examples, the interface 700 may output vessel data, such as EEL value, lumen values, VFR values, MLA values, percent diameter stenosis, or the like. The interface 700 may include a pre-stent value and a predicted post-stent value. For example, the pre-stent VFR value may be 0.75 whereas the predicted post stent VFR value may be 0.90. Similarly, while not shown, the interface 700 may provide for output a pre-stent EEL value, lumen diameter value, or the like and a corresponding predicted post-stent value. The predicted values may be determined using machine learning or neural networks that have been trained for predictive analysis.

FIG. 8 depicts a flow diagram of an example process for automatically determining a plaque burden for frames within a region of interest, which can be implemented using the computing device 112. The following operations do not have to be performed in the precise order described below. Rather, various operations can be handled in a different order or simultaneously, and operations may be added or omitted.

In block 810, vessel data comprising intravascular imaging data may be received. The intravascular imaging data may be, for example, one or more image frames obtained by a probe during a pullback through a vessel. The one or more image frames may be OCT image frames, IVUS image frames, or the like. The vessel data may be, in some examples, values or measurements associated with the vessel in a given image frame. For example, the vessel data may be EEL values, such as an EEL diameter, lumen values, such as the lumen area or diameter, or the like. In some examples, the vessel data may include pressure values, VFR value, IMR values, or the like.

In block 820, a region of interest of a vessel may be identified based on the vessel data. For example, the vessel data may include indications of one or more side branches. The region of interest may be defined as a region between a first side branch and a second side branch.

In block 830, first subset of frames within the region of interest having a plaque burden below a threshold plaque burden may be identified. The threshold plaque burden may be, for example, between 0-45%. The first subset of frames may include a threshold number of frames. The threshold number of frames may be at least three frames.

In block 840, an EEL value may be determined for the first subset of frames. According to some examples, the EEL value may be determined for each frame of the first subset of frames. In another example, the EEL value may be determined for a threshold number of frames of the first subset of frames. The EEL value for the first subset of frames may be automatically determined based on the vessel data. The EEL value may be, for example, an EEL diameter and/or EEL area.

In block 850, a representative EEL value for the region of interest may be determined based on the EEL values of the first subset of frames. The representative EEL value for the region of interest may be, for example, a median EEL value, an average EEL value, a mean EEL value, and/or a mode EEL value. For example, the median of the EEL values of the first subset of frames may be determined. The determined representative EEL value of the first subset of frames may be used as the predicted EEL value for a second subset of frames in the region of interest. According to some examples, the determined representative EEL value of the first set of frames may be used as the predicted EEL value for some of the frames in the first subset of frames. For example, if some of the frames in the first subset of frames have erroneously measured EEL values, the determined representative EEL value may be used as the predicted EEL value for the frames in the first subset of frames having an erroneous EEL value. The erroneous EEL value may be an EEL value that is beyond a standard deviation of the determined representative EEL value, that is outside a predetermined range of the determined representative EEL value, or the like.

The second subset of frames may be different than the first subset of frames. The second subset of frames may be frames within the region of interest in which an EEL value was not automatically determined at the time of the pullback. In some examples, the second subset of frames may be frames within the region of interest in which the EEL value of those frames was erroneously measured, not visible, etc.

In block 860, a plaque burden for frames within the region of interest may be determined based on the representative EEL value. The plaque burden may be further based on a lumen area for the respective frame. For example, the plaque burden may be determined on a per frame basis using the equation: 1−(lumen area/representative EEL value). In this equation, the representative EEL value may be the representative EEL area and/or representative EEL diameter.

According to some examples, the plaque burden for each frame may be used to identify lesions within the vessel. For example, the lesions may be identified by identifying the image frame with a maximum plaque burden. According to some examples, the lesions may be identified by identifying the image frames with a plaque burden greater than a disease threshold. The disease threshold may be, for example, a plaque burden disease threshold. The plaque burden disease threshold may be, for example, approximately 70%. In some examples, the plaque burden disease threshold may be greater or less than 70%. For example, the plaque burden disease threshold may be patient specific, determined by the physician, or the like.

Using the identified image frame as the initial frame, the plaque burden of frames proximal and distal to the initial frame may be compared to a treatment zone threshold until an image frame proximal and distal to the initial frame has a plaque burden less than the treatment zone threshold. The treatment zone threshold may, in some examples, be more or less than a plaque burden of 50%. For example, the treatment zone threshold may correspond to a plaque burden of approximately 45%. In some examples, the treatment zone threshold may be a range, such as a plaque burden of approximately 0-50%. In some examples, the treatment zone threshold may be patient specific, determined by the physician, etc. The treatment zone end frames may, additionally or alternatively, correspond to a frame with a visible EEL arc greater than or equal to an arc threshold. According to some examples, the arc threshold may be at least or about 180 degrees. In some examples, the arc threshold may be more or less than 180 degrees. For example, the arc threshold may be patient specific, determined by a physician, or the like such that the arc threshold can be any predetermined value.

The image frames between the identified proximal and distal frame are then marked as corresponding to a lesion. The process is repeated for the image frame having the next greatest plaque burden, without considering the image frames that have been identified as corresponding to a lesion. After each lesion is identified, the gap, or distance, between lesions is determined. In examples where the gap is substantially equal to or less than a distance threshold, the lesions may be combined and identified as a single lesion. For example, when a first lesion is within a minimum gap distance from a second lesion, e.g., the gap between the first and second lesion is less than the distance threshold, the first and second lesion may be identified as a combined lesion. In examples where the gap is greater than the distance threshold, the lesions may be identified as individual lesions. According to some examples, suggested, or candidate, treatment zones for a stent may be identified based on the identified lesions.

FIG. 9 depicts a flow diagram of an example process for automatically determining a plaque burden for frames within a region of interest, which can be implemented using the computing device 112. The following operations do not have to be performed in the precise order described below. Rather, various operations can be handled in a different order or simultaneously, and operations may be added or omitted.

In block 910, vessel data comprising intravascular imaging data may be received. The intravascular imaging data may be, for example, one or more image frames obtained by a probe during a pullback through a vessel. The one or more image frames may be OCT image frames, IVUS image frames, or the like. The vessel data may be, in some examples, values or measurements associated with the vessel in a given image frame. For example, the vessel data may be EEL values, such as an EEL diameter, lumen values, such as the lumen area or diameter, or the like. In some examples, the vessel data may include pressure values, VFR value, IMR values, or the like.

In block 920, a region of interest of a vessel may be identified based on the vessel data. For example, the vessel data may include indications of one or more side branches. The region of interest may be defined as a region between a first side branch and a second side branch.

In block 930, a predicted EEL value for frames within the region of interest may be determined as a function of intima thickness and media thickness. According to some examples, the predicted EEL value may be a representative EEL value for the frames within the region of interest. In another example, the predicted EEL value may be determined on a per frame basis for frames within the region of interest. For example, for each frame within the region of interest, a predicted EEL value may be determined. In some examples, a respective predicted EEL value may be determined for at least some of the frames within the region of interest. According to some examples, the intima thickness may be a minimum intima thickness and the media thickness may be a minimum media thickness. The minimum intima thickness and minimum media thickness may be determined based on histologic observations vessels. For example, based on histologic observations of vessel, an intima thickness and media thickness may be determined with a standard deviation. The minimum intima thickness and minimum media thickness may be determined based on the low end of the standard deviation.

According to some examples, the predicted EEL may be further determined as a function of a H-K model. The H-K model predicts the lumen diameter based on the assumption that the diameter, circumference, and/or area of the vessel is substantially constant along the region of interest. For example, the predicted EEL may be determined using the following equation:

$$Predicted EEL value = ( Expected Lumen Diameter ) + 2 * ( Minimum Intima Thickness + Minimum Media Thickness )$$

In the equation, the predicted EEL value may be the predicted EEL area and/or predicted EEL diameter. The expected lumen diameter may be, for example, the lumen diameter determined using the H-K model.

According to some examples, the predicted EEL value, determined as a function of the H-K model, may be compared to an EEL value of a proximal segment of the vessel and/or a distal segment of the vessel. The EEL value of the proximal segment and/or the distal segment of the vessel may be, in some examples, a representative EEL value of the proximal and/or distal segment of the vessel. The representative EEL value for the region of interest may be, for example, a median EEL value, an average EEL value, a mean EEL value, and/or a mode EEL value. If the predicted EEL value is less than the representative EEL value of the distal segment, than the representative EEL value of the distal segment may be used to determine the plaque burden of frames within the region of interest. The representative EEL value of the distal segment may be used when the predicted EEL value is less than the representative EEL value of the distal segment due to the H-K model assuming that the vessel has a natural taper from a proximal vessel segment to a distal vessel segment. If the predicted EEL value is greater than the representative EEL value of the proximal segment, the representative EEL value of the proximal segment may be used to determine the plaque burden of the frames within the region of interest. The representative EEL value of the proximal segment may be used when the predicted EEL value is greater than the representative EEL value of the proximal segment due to the H-K model assuming that the vessel has a natural taper from a proximal vessel segment to a distal vessel segment.

In block 940, a plaque burden for frames within the region of interest may be determined based on the predicted EEL value. The plaque burden may be further based on a lumen area for the respective frame. For example, the plaque burden may be determined on a per frame basis using the equation: 1−(lumen area/predicted EEL value).

Similar to the example process described with respect to FIG. 8, the plaque burden for each frame may be used to identify lesions within the vessel. For example, the lesions may be identified by identifying the image frame with a maximum plaque burden. According to some examples, the lesions may be identified by identifying the image frames with a plaque burden greater than a disease threshold. The disease threshold may be, for example, a plaque burden disease threshold. The plaque burden disease threshold may be, for example, approximately 70%. In some examples, the plaque burden disease threshold may be greater or less than 70%. For example, the plaque burden disease threshold may be patient specific, determined by the physician, or the like.

Using that image frame as the initial frame, the plaque burden of frames proximal and distal to the initial frame may be compared to a treatment zone threshold until an image frame proximal and distal to the initial frame has a plaque burden less than the treatment zone threshold and/or the arc of visible EEL is above an arc threshold. The image frames between the identified proximal and distal frame are then marked as corresponding to a lesion. The process is repeated for the image frame having the next greatest plaque burden, without considering the image frames that have been identified as corresponding to a lesion. After each lesion is identified, the gap, or distance, between lesions is determined. In examples where the gap is substantially equal to or less than a distance threshold, the lesions may be combined and identified as a single lesion. For example, when a first lesion is within a minimum gap distance from a second lesion, e.g., the gap between the first and second lesion is less than the distance threshold, the first and second lesion may be identified as a combined lesion. In examples where the gap is greater than the distance threshold, the lesions may be identified as individual lesions. According to some examples, suggested, or candidate, treatment zones. According to some examples, the suggested, or candidate, treatment zone may be a stent landing zone. The stent landing zone for a stent may be identified based on the identified lesions. In some examples, the treatment zone may be for a balloon angioplasty, laser atherectomy, or the like.

According to some examples, the EEL values for a given frame during a pullback may be undeterminable due to plaque overgrowth or other impediments during the pullback. In such examples, predicting the EEL values for those image frames may provide for more vessel information to be determined. For example, by predicting the EEL values either by averaging EEL values or as a function of intima thickness, media thickness, and the H-K model, a plaque burden may be determined on a frame-by-frame basis. The determine plaque burden may be used to more accurately identify lesion locations, lengths, severity, or the like. Moreover, by using the predicted EEL values to determine the plaque burden for frames where EEL was not detected or incorrectly measured, candidate treatment zones may be more accurately identified. For example, candidate treatment zones are typically adjusted by physicians during the stent planning phase. In contrast, by automatically determining, or predicting, EEL values and using the determined EEL values to determine plaque burden, candidate treatment zone end frames may be automatically identified by identifying locations with a plaque burden below a threshold and/or an visible EEL arc at or above an arc threshold. This mitigates the need for user intervention and allows for greater accuracy as the predicted EEL values provide for a more informed decision making process.

In general, the systems and methods disclosed herein provide to various automated clinical tools to help physicians determine if they should treat a given patient and, if so, which lesion/stenosis should be treated. The system may provide guidance to treat the most significant lesion based on physiology. In addition, the details relating to plaque type, and other MLS detected features can be used to selected shortest stent that provides maximal flow recovery. In addition, virtual-stenting can be implemented that provides interactive planning that allows for stent to be tailored for placement in an artery that is informed by tissue classifications and other measurements as disclosed herein.

The aspects, features, and examples of the disclosure are to be considered illustrative in all respects and are not intended to limit the disclosure, the scope of which is defined only by the claims. Other examples, modifications, and usages will be apparent to those skilled in the art without departing from the spirit and scope of the claimed invention.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise. As used herein, the term “about” refers to a ±10% variation from the nominal value. All numerical values and ranges disclosed herein are deemed to include “about” before each value.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

1. A method, comprising:
receiving, by one or more processors, vessel data comprising intravascular imaging data;
identifying, by the one or more processors based on the vessel data, a region of interest of a vessel;
identifying, by the one or more processors, a first subset of frames within the region of interest having a plaque burden below a threshold plaque burden, the first subset of frames including at least a threshold number of frames;
determining, by the one or more processors, an external elastic lamina (“EEL”) value for the first subset of frames;
determining, by the one or more processors based on the EEL values of the first subset of frames, a representative EEL value for the region of interest; and
determining, by the one or more processors based on the representative EEL value, the plaque burden for a second subset of frames within the region of interest, wherein the second subset of frames is different than the first subset of frames.

2. The method of claim 1, wherein the representative EEL value is at least one of a mean EEL value, an average EEL value, a median EEL value, or a mode EEL value.

3. The method of claim 1, wherein the region of interest is defined between a first vessel branch and a second vessel branch.

4. The method of claim 1, wherein:
the EEL value is an EEL diameter or an EEL area, and
the representative EEL value is a representative EEL diameter or a representative EEL area.

5. The method of claim 1, wherein the threshold plaque burden corresponds to a plaque burden at or about 0-50%.

6. The method of claim 1, wherein the threshold number of frames is at least three frames.

7. The method of claim 1, wherein determining the plaque burden is further based on a lumen area for a respective frame.

8. The method of claim 76, wherein determining the plaque burden for the respective frame comprises calculating as 1−(the lumen area/the representative EEL value).

9. The method of claim 1, further comprising identifying, by the one or more processors based on the determined plaque burden, lesions.

10. The method of claim 9, further comprising identifying, by the one or more processors based on the identified lesions, a candidate treatment zone.

11. The method of claim 10, wherein identifying the candidate treatment zone further comprises identifying, by the one or more processors, frames proximal and distal to the identified lesions, wherein at least one of the plaque burden of the frames proximal and distal to the identified lesions is less than a threshold or a visible EEL arc is greater than or equal to an arc threshold.

12. The method of claim 9, wherein when a first lesion is within a minimum gap distance from a second lesion, the method further comprises identifying, by the one or more processors, the first lesion and the second lesion as a combined lesion.

13. A system, comprising:
one or more processors, the one or more processors configured to:
receive vessel data comprising intravascular imaging data;
identify, based on the vessel data, a region of interest of a vessel;
identify a first subset of frames within the region of interest having a plaque burden below a threshold plaque burden, the first subset of frames including at least a threshold number of frames;
determine an external elastic lamina (“EEL”) value for the first subset of frames;
determine, based on the EEL values of the first subset of frames, a representative EEL value for the region of interest; and
determine, based on the representative EEL value, a plaque burden for a second subset of frames within the region of interest, wherein the second subset of frames is different than the first subset of frames.

14. The system of claim 13, wherein at least one of:
the representative EEL value is at least one of a mean EEL value, an average EEL value, a median EEL value, or a mode EEL value, or
the region of interest is defined between a first vessel branch and a second vessel branch.

15. (canceled)

16. The system of claim 13, wherein:
the EEL value is an EEL diameter or an EEL area, and
the representative EEL value is a representative EEL diameter or a representative EEL area.

17. The system of claim 13, wherein at least one of:
the threshold plaque burden corresponds to a plaque burden at or about 0-50%, or
the threshold number of frames is at least three frames.

18. (canceled)

19. The system of claim 13, wherein determining the plaque burden is further based on a lumen area for a respective frame.

20. (canceled)

21. The system of claim 13, wherein the one or more processors are further configured to:
identify, based on the determined plaque burden, lesions; and
identify, when a first lesion is within a minimum gap distance from a second lesion, the first lesion and the second lesion as a combined lesion.

22. The system of claim 13, wherein the one or more processors are further configured to;
identify, based on the determined plaque burden, lesions;
identify, based on the identified lesions, a candidate treatment zone;
identify frames proximal and distal to the identified lesions, wherein at least one of the plaque burden of the frames proximal and distal to the identified lesions is less than a threshold or a visible EEL arc is greater than or equal to an arc threshold.

23. (canceled)

24. (canceled)

25. A non-transitory computer-readable medium storing instructions, which when executed by one or more processors, cause the one or more processors to:
receive vessel data comprising intravascular imaging data;
identify, based on the vessel data, a region of interest of a vessel;
identify a first subset of frames within the region of interest having a plaque burden below a threshold plaque burden, the first subset of frames including at least a threshold number of frames;
determine an external elastic lamina (“EEL”) value for the first subset of frames;
determine, based on the EEL values of the first subset of frames, a representative EEL value for the region of interest; and
determine, based on the representative EEL value, a plaque burden for a second subset of frames within the region of interest, wherein the second subset of frames is different than the first subset of frames.

26-36. (canceled)

37. A method, comprising:
receiving, by one or more processors, vessel data comprising intravascular imaging data;
identifying, by the one or more processors based on the vessel data, a region of interest of a vessel;
determining, by the one or more processors based on the vessel data, a predicted elastic external lamina (“EEL”) value for frames within the region of interest as a function of intima thickness and media thickness; and
determining, by the one or more processors based on the predicted EEL value, a plaque burden for frames within the region of interest.

38. The method of claim 37, wherein the region of interest is defined between a first vessel branch and a second vessel branch.

39. The method of claim 37, wherein the predicted EEL value is further determined as a function of at least one of a lumen area or a lumen diameter derived from a H-K model.

40. The method of claim 37, wherein determining the plaque burden is further based on a lumen area for a respective frame.

41. The method of claim 40, wherein determining the plaque burden for the respective frame includes calculating 1−(the lumen area/the predicted EEL value).

42. The method of claim 37, further comprising identifying, by the one or more processors based on the determined plaque burden, lesions.

43. The method of claim 42, further comprising identifying, by the one or more processors based on the identified lesions, a candidate treatment zone.

44. The method of claim 43, wherein identifying the candidate treatment zone further comprises identifying, by the one or more processors, frames proximal and distal to the identified lesions, wherein at least one of the plaque burden of the frames proximal and distal to the identified lesions is less than a threshold or a visible EEL arc is greater than or equal to an arc threshold.

45. The method of claim 44, wherein when a first lesion is within a minimum gap distance from a second lesion, the method further comprises identifying, by the one or more processors, the first lesion and the second lesion as a combined lesion.

46. The method of claim 37, further comprising:
comparing, by the one or more processors, the predicted EEL value for the region of interest to a representative EEL value for a proximal segment of the vessel; and
when the representative EEL value for the proximal segment is less than the predicted EEL value for the region of interest, determining the plaque burden for the region of interest is based on the representative EEL value of the proximal segment.

47. The method of claim 37, further comprising:
comparing, by the one or more processors, the predicted EEL value for the region of interest to a representative EEL value for a distal segment of the vessel; and
when the representative EEL value for the distal segment is greater than the predicted EEL value for the region of interest, determining the plaque burden for the region of interest is based on the representative EEL value of the distal segment.

48. The method of claim 46, wherein the representative EEL value is at least one of a mean EEL value, an average EEL value, a median EEL value, or a mode EEL value.

49-72. (canceled)
